(19) United States
(12) Patent Application Publication
(10) Pub. No.: US 2026/0015653 A1
(43) Pub. Date: Jan. 15, 2026

(54) METHODS FOR INCREASING RESOLUTION OF SPATIAL ANALYSIS

(71) Applicant: 10X Genomics, Inc., Pleasanton, CA (US)

(72) Inventors: Jennifer Chew, Yayward, CA (US); Yifengq Yin, Elk Grove3, CA (US); Layla Katiraee, Castro Valley, CA (US)

(21) Appl. No.: 19/274,921

(22) Filed: Jul. 21, 2025

Related U.S. Application Data
(62) Division of application No. 17/971,174, filed on Oct. 21, 2022, now Pat. No. 12,365,935.
Application No. 17/971,174 is a continuation of application No. PCT/US2022/028071, filed on May 6, 2022.
(60) Provisional application No. 63/185,035, filed on May 6, 2021.

(51) Int. Cl. C12Q 1/6841; C12Q 1/6806; C12Q 1/6844

(52) U.S. Cl. CPC C12Q 1/6841; C12Q 1/6806; C12Q 1/6844; C12Q 2600/16

(57) ABSTRACT

Provided herein are methods for capturing an analyte from a first region of interest of a biological sample on a substrate, where the biological sample comprises the first region of interest and a second region, and where the method includes contacting the second region with a sealant in order to create a hydrophobic seal thereby preventing an interaction between an analyte from the second region with a capture domain of a capture probe.

### CROSS-REFERENCE TO RELATED APPLICATIONS

This application is a divisional application of U.S. application Ser. No. 17/971,174, filed on Oct. 21, 2022, which pursuant to 35 U.S.C. § 119(e), is a continuation of International Application PCT/US2022/028071, with an international filing date of May 6, 2022, which claims the benefit to U.S. Provisional Patent Application No. 63/185,035, filed May 6, 2021. The contents of this priority application are incorporated herein by reference in their entireties.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Increasing resolution of spatial heterogeneity can be achieved by selectively analyzing areas of interest on a substrate or in a biological sample. This is usually achieved by mechanically or enzymatically removing or ablating non-areas of interest, both of which can damage the analytes of interest. Therefore, alternative methods are needed that better preserve the captured analytes or decrease the amount of data generated (and thus resources used) for biological samples that are not of interest.

### SUMMARY

Capturing analytes from a region of interest while excluding analytes from regions that are not currently of interest would be beneficial in a number of ways. For example, if a researcher stains a tissue to determine that there is one or a few regions of interest in a tissue that are of interest (e.g., not the whole tissue is of interest) it would be advantageous to look at only that one or a few regions of interest. Further, a researcher may be interested in only a specific portion, side, middle, edges, etc. of a tissue, but it is very difficult to isolate and assay those areas from the main tissue. It would be advantageous to have a method for isolating regions of interest, potentially interrelated areas of interest, from a whole tissue where differentially sectioning a tissue to isolate those areas is not always possible. As such, provided herein are methods for capturing an analyte from a first region of interest of a biological sample on a substrate, where the biological sample comprises the first region of interest and a second region, and where the method includes contacting the second region with a sealant in order to create a hydrophobic seal thereby preventing an interaction between an analyte from the second region with a capture domain of a capture probe.

Surprisingly, the methods described here also have the advantage of increasing the resolution of the spatial array of the regions of interest. As such, some embodiments of the methods result in greater sequencing depth, greater number of reads per spot, and greater number of unique molecular identifiers (UMIs) per spot for the region of interest as compared to a biological sample not contacted with a sealant. Therefore, the methods described herein further increase resolution of spatial analysis methodologies described herein and known in the art.

Some embodiments of the methods result in greater sequencing depth for the biological sample regions of interest as compared to the capture efficiency and sequencing depth for a biological sample not contacted with a sealant. Some embodiments of these methods result in a greater number of reads per spot for the biological sample as compared to the number of reads per spot for a biological sample not contacted with a sealant. Some embodiments of any of these methods result in a greater number of unique molecule identifier (UMI) counts per spot for the biological sample as compared to the number of UMI counts per spot for a biological sample not contacted with a sealant. As such, practicing the methods described herein not only allow for isolation of a region of interest for a more focused transcriptomic study, but they also provide the additional benefit of increasing the resolution for that region of interest being assayed.

In one aspect, provided herein is a method for capturing an analyte from a first region of interest of a biological sample, the method comprising: (a) contacting the biological sample with a substrate comprising a plurality of capture probes, wherein the capture probe comprises a spatial barcode and a capture domain; (b) identifying the first region of interest in the biological sample and a second region; (c) contacting the second region with a sealant; and (d) hybridizing the analyte from the first region of interest to the capture probe, thereby capturing the analyte from the first region of interest of the biological sample. In some instances, the sealant generates a hydrophobic seal that covers the second region, thereby preventing an interaction between an analyte from the second region and the capture domain of the capture probe.

In some instances, disclosed herein is a method for capturing an analyte from a first region of interest of a biological sample, comprising, (a) contacting the biological sample with a substrate comprising a plurality of capture probes, wherein the capture probe comprises a spatial barcode and a capture domain, (b) determining a first region of interest in on the biological sample and a second region and contacting the second region with a sealant, and (c) hybridizing the analyte from the first region of interest to the capture probe, thereby capturing the analyte from the first region of interest of the biological sample. In some instances, the sealant generates a hydrophobic seal that covers the second region, thereby preventing an interaction between an analyte from the second region and the capture domain of the capture probe.

In another aspect, provided herein is a method for capturing an analyte from a first region of interest of a biological sample, comprising, (a) providing a biological sample comprising: (i) the first region of interest, and (ii) the second region covered with a sealant, wherein the sealant generates a hydrophobic seal that covers the second region, thereby preventing an interaction between an analyte from the second region with a capture domain of a capture probe; and (b) contacting the biological sample with a substrate comprising a plurality of capture probes, wherein the capture probe comprises a spatial barcode and a capture domain, and (c) hybridizing the analyte from the first region of interest to the capture probe, thereby capturing the analyte from the first region of interest of the biological sample.

In some embodiments of any of the methods described herein, the methods further comprise determining (i) all or a portion of a sequence corresponding to the analyte bound to the capture domain or a complement thereof, and (ii) the sequence corresponding to the spatial barcode or a complement thereof.

In some embodiments, the method further comprises using the determined sequences of (i) and (ii) to determine the abundance or location of the analyte in the biological sample. In some embodiments of any of the methods described herein, further comprise identifying the first region of interest and the second region for applying a sealant prior to step (a).

In some embodiments, the first region of interest and second region are identified by a tissue detection machine learning module. In some embodiments, the contacting in step (b) comprises applying the sealant using a method selected from: a brush tip, a dropper, a pipette, a microfluidic device, and a liquid handling instrument. In some embodiments, the applying the sealant comprises automation. In some embodiments, the applying the sealant comprises identifying the first region of interest and the second region and applying the sealant using automation to the second region. In some embodiments, the automation comprises a system comprising a computer implemented method and a liquid handling instrument.

In some embodiments of any of the methods described herein, the sealant comprises a sealant that is capable of generating a hydrophobic seal. In some embodiments, the sealant is selected from the group consisting of: a coverslip sealant, a liquid coverslip, liquid from a hydrophobic pen, a mounting media, a gel, an adhesive, and a bilayer, or a combination In some embodiments, the sealant comprises a coverslip sealant. In some thereof. embodiments, the coverslip sealant is Covergrip™ Coverslip Sealant. In some embodiments of any of the methods described herein, further comprising allowing a period of time for the sealant to form a hydrophobic seal. In some embodiments, the period of time comprises about 15 minutes to about 4 hours. In some embodiments, the period of time is about 1 hour.

In some embodiments of any of the methods described herein, the methods further comprise contacting the biological sample with a permeabilization agent, wherein the permeabilization agent is selected from an organic solvent, a detergent, and an enzyme, or a combination thereof.

In some embodiments, the permeabilization agent is selected from the group consisting of: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™. In some embodiments, the endopeptidase is pepsin or proteinase K. In some embodiments, the permeabilizing step is performed after contacting the biological sample with the substrate.

In some embodiments of any of the methods described herein, further comprise removing the sealant from the biological sample. In some embodiments, removing comprises lifting, peeling, dissolving, liquefying, or decrosslinking the sealant from the biological sample. In some embodiments, removing comprises contacting the biological sample with a removing agent selected from the group consisting of: a solvent, an acid, a base, and a buffer, or any combinations thereof.

In some embodiments, the removing agent comprises phosphate buffered saline (PBS). In some embodiments, removing comprises contacting the sealant with PBS. In some embodiments of any of the methods described herein, the biological sample is a tissue sample. In some embodiments, the tissue sample is a formalin-fixed, paraffin-embedded (FFPE) tissue sample, a fresh tissue sample, or a frozen tissue sample. In some embodiments, the tissue sample is the FFPE tissue sample, and the tissue sample is decrosslinked.

In some embodiments of any of the methods described herein, the biological sample was previously stained. In some embodiments, the biological sample was previously stained using immunofluorescence or immunohistochemistry. In some embodiments, the biological sample was previously stained using hematoxylin and eosin.

In some embodiments of any of the methods described herein, the method results in greater capture efficiency for the biological sample as compared to the capture efficiency for a biological sample not contacted with a sealant. In some embodiments of any of the methods described herein, the second region is a region on the biological sample. In some embodiments, the second region is a region surrounding the biological sample on the substrate. In some embodiments, the second region comprises a region on the biological sample and a region surrounding the biological sample.

In another aspect, provided herein is a kit comprising: (a) a sealant; (b) a substrate comprising a plurality of capture probes, wherein the capture probes comprises a spatial barcode and a capture domain; and (c) instructions for performing the method of any one of the preceding claims. In some embodiments, the sealant comprises a coverslip sealant. In some embodiments, the coverslip sealant is Covergrip™ Coverslip Sealant. In some embodiments, the kit further comprises a sealant applicator.

In another aspect, disclosed herein is a composition comprising: (a) an array, wherein the array comprises a plurality of capture probes and wherein a capture probe of the plurality comprises a spatial barcode and a capture domain, (b) a biological sample positioned on the array, and (c) a sealant applied to a portion of the biological sample.

In another aspect, disclosed herein is a composition comprising, (a) an array, wherein the array comprises a plurality of capture probes and wherein a capture probe of the plurality comprises a spatial barcode and a capture domain, (b) a biological sample positioned on the array, and (c) a sealant applied to the area on the array surrounding the biological sample.

In another aspect, disclosed herein is a composition comprising, (a) an array, wherein the array comprises a plurality of capture probes and wherein a capture probe of the plurality comprises a spatial barcode and a capture domain, (b) a biological sample positioned on the array, and (c) a sealant applied to a portion of the biological sample and the area on the array surrounding the biological sample. In some embodiments, a plurality of analytes from the biological sample hybridize to capture domains of capture probes located under the biological sample and do not substantially hybridize or do not hybridize to capture domains of the capture probes under the sealant.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow for evaluating analytes in a region of interest using a sealant.

FIG. 2 is a schematic diagram showing an example of a capture probe, as described herein.

FIG. 3 is a schematic illustrating a cleavable capture probe, wherein the cleaved capture probe can enter into a cell and bind to target analytes within the sample.

FIG. 4 is a schematic diagram of an exemplary multiplexed spatially-barcoded feature.

FIG. 5 is a schematic showing the exemplary arrangement of barcoded features on a substrate, generating an arrayed substrate.

FIG. 6 shows the areas of the biological sample to which a sealant was applied as indicated by the solid black line.

FIG. 7 shows gene expression heat maps for the biological samples from FIG. 6. Area of the sealant is indicated by the solid while line in the right images.

FIG. 8 shows fraction of raw reads on target and unambiguously mapped (bottom panels), and fractions of spots under tissue (top panels) of liver (left panels) and spleen (right panels) biological samples were positioned on a substrate with an area outside of the biological sample was unblocked (control), blocked with cover grip sealant, or blocked with nail polish. Each spot is representative of a biological sample.

FIG. 9 shows fraction of reads in spots under the tissue (bottom panels), and fraction of targeted reads useable (top panels) of liver (left panels) and spleen (right panels) biological samples were positioned on a substrate with an area outside of the biological sample was unblocked (control), blocked with cover grip sealant, or blocked with nail polish.

FIG. 10 shows median panel genes detected at 10,000 panel reads per spot (bottom panels) and median panel reads detected at 2,500 panel reads per spot (top panels) of liver (left panels) and spleen (right panels) biological samples were positioned on a substrate with an area outside of the biological sample was unblocked (control), blocked with cover grip sealant, or blocked with nail polish.

FIG. 11A shows median panel UMI counts at 10,000 panel reads per spot (bottom panels) and median panel UMI counts at 5,000 panel reads per spot (top panels) of liver (left panels) and spleen (right panels) biological samples were positioned on a substrate with an area outside of the biological sample was unblocked (control), blocked with cover grip sealant, or blocked with nail polish.

FIG. 11B shows median panel UMI counts at 10000 raw reads per spot (bottom panels) and median panel UMI counts at 1,000 raw reads per spot (top panels) of liver (left panels) and spleen (right panels) biological samples were positioned on a substrate with an area outside of the biological sample was unblocked (control), blocked with cover grip sealant, or blocked with nail polish.

FIG. 12A shows panel cDNA PCR duplication of 5,000 panel reads per spot (bottom panels) and panel cDNA PCR duplication of 1,000 panel reads per spot (top panels) of liver (left panels) and spleen (right panels) biological samples were positioned on a substrate with an area outside of the biological sample was unblocked (control), blocked with cover grip sealant, or blocked with nail polish.

FIG. 12B shows panel cDNA PCR duplication of 5,000 raw reads per spot (bottom panels) and panel cDNA PCR duplication of 2,500 raw reads per spot (top panels) of liver (left panels) and spleen (right panels) biological samples were positioned on a substrate with an area outside of the biological sample was unblocked (control), blocked with cover grip sealant, or blocked with nail polish.

FIG. 13A shows spatial sequencing analysis of spleen and liver biological samples uncovered (control), blocked with cover grip sealant, or blocked with nail polish.

FIG. 13B shows a tissue plot clustering of transcripts of spleen and liver biological samples uncovered (control), blocked with cover grip sealant, or blocked with nail polish.

### DETAILED DESCRIPTION

### I. Introduction

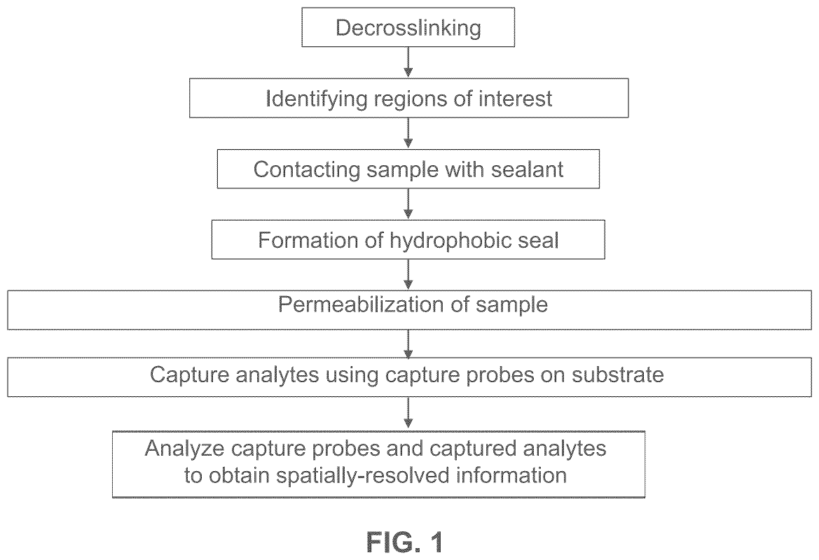

Provided herein are methods for capturing an analyte from a region of interest of a biological sample on a substrate, where the biological sample comprises the first region that is the region of interest and a second region, and where the method includes contacting the second region with a sealant in order to create a hydrophobic seal thereby preventing an interaction between an analyte in the second region with a capture domain of a capture probe (see FIG. 1). Some embodiments of the methods result in greater sequencing depth for the region of interest in the biological sample as compared to the capture efficiency and sequencing depth for a biological sample not contacted with a sealant. Some embodiments of these methods result in a greater number of reads per spot for the biological sample as compared to the number of reads per spot for a biological sample not contacted with a sealant. Some embodiments of any of these methods result in a greater number of unique molecule identifier (UMI) counts per spot for the biological sample as compared to the number of UMI counts per spot for a biological sample not contacted with a sealant.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., WO 2020/176788 and/or U.S. Patent Application Publication No. 2020/0277663. Generation of capture probes can be achieved by any appropriate method, including those described in WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in WO 2020/176788 and/or U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). In some instances, the spatially-barcoded array populated with capture probes (as described further herein) is contacted with a biological sample, and the biological sample is permeabilized, allowing the analyte to migrate away from the sample and toward the array. The analyte interacts with a capture probe on the spatially-barcoded array. Another method is to cleave spatially-barcoded capture probes from an array and promote the spatially-barcoded capture probes towards and/or into or onto the biological sample. In some instances, the spatially-barcoded array populated with capture probes (as described further herein) can be contacted with a sample. The spatially-barcoded capture probes are cleaved and then interact with cells within the provided biological sample. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell.

In some instances, sample preparation may include placing the sample on a slide, fixing the sample, and/or staining the biological sample for imaging. The stained sample can be then imaged on the array using both brightfield (to image the sample hematoxylin and eosin stain) and/or fluorescence (to image features) modalities. Optionally, the sample can be destained prior to permeabilization. In some embodiments, analytes are then released from the sample and capture probes forming the spatially-barcoded array hybridize or bind the released analytes. The sample is then removed from the array and the capture probes cleaved from the array. The biological sample and array are then optionally imaged a second time in one or both modalities while the analytes are reverse transcribed into cDNA, and an amplicon library is prepared and sequenced. Images are then spatially-overlaid in order to correlate spatially-identified biological sample information. When the sample and array are not imaged a second time, a spot coordinate file is supplied instead. The spot coordinate file replaces the second imaging step. Further, amplicon library preparation can be performed with a unique PCR adapter and sequenced.

In some instances, disclosed is an exemplary workflow that utilizes a spatially-barcoded array on a substrate, where spatially-barcoded capture probes are clustered at areas called features. The spatially-barcoded capture probes can include a cleavage domain, one or more functional domains, a spatial barcode, a unique molecular identifier, and a capture domain. The spatially-barcoded capture probes can also include a 5′ end modification for reversible attachment to the substrate. The spatially-barcoded array is contacted with a biological sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The biological sample can be optionally removed from the array.

The capture probes can be optionally cleaved from the array, and the captured analytes can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme in a template independent manner. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be amplified using PCR, where the forward and reverse primers flank the spatial barcode and analyte regions of interest, generating a library associated with a particular spatial barcode. In some embodiments, the library preparation can be quantitated and/or quality controlled to verify the success of the library preparation steps. In some embodiments, the cDNA comprises a sequencing by synthesis (SBS) primer sequence. The library amplicons are sequenced and analyzed to decode spatial information.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some instances, the capture probe can include functional sequences that are useful for subsequent processing. In some instances, a capture probe can be reversibly attached to a substrate via a linker. The capture probe can include one or more functional sequences, which can include a sequencer specific flow cell attachment sequence, e.g., a P5 or P7 sequence, as well as functional sequence, which can include sequencing primer sequences, e.g., a R1 primer binding site, a R2 primer binding site. In some embodiments, sequence is a P7 sequence and sequence is a R2 primer binding site. A capture probe can additionally include a spatial barcode and/or unique molecular identifier and a capture domain. The different sequences of the capture probe need not be in the sequential manner as depicted in this example, however the capture domain should be placed in a location on the barcode wherein analyte capture and extension of the capture domain to create a copy of the analyte can occur.

In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

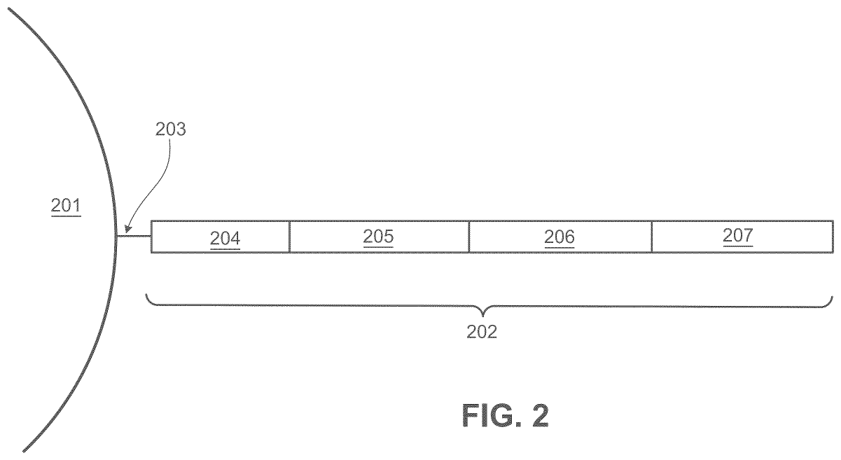

FIG. 2 is a schematic diagram showing an exemplary capture probe, as described herein. As shown, the capture probe 202 is optionally coupled to a feature 201 by a cleavage domain 203, such as a disulfide linker. The capture probe can include a functional sequence 204 that are useful for subsequent processing. The functional sequence 204 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 205. The capture probe can also include a unique molecular identifier (UMI) sequence 206. While FIG. 2 shows the spatial barcode 205 as being located upstream (5′) of UMI sequence 206, it could also be reversed such that the spatial barcode could be upstream of the UMI. The capture probe also includes a capture domain 207 to facilitate capture of a target analyte. In some embodiments, the capture probe comprises one or more additional functional sequences that can be located, for example between the spatial barcode 205 and the UMI sequence 206, between the UMI sequence 206 and the capture domain 207, or following the capture domain 207. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

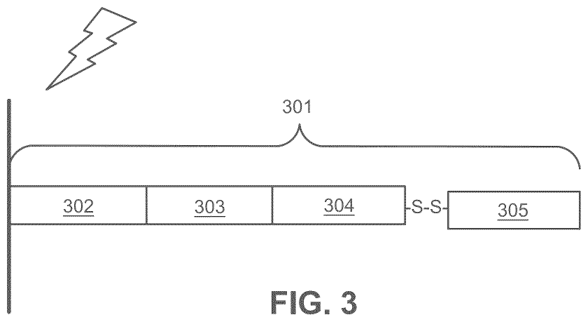

In some cases, capture probes are introduced into the cell using a cell-penetrating peptide. FIG. 3 is a schematic illustrating a cleavable capture probe that includes a cell-penetrating peptide, wherein the cleaved capture probe can enter into a non-permeabilized cell and bind to analytes within the sample. The capture probe 301 contains a cleavage domain 302, a cell penetrating peptide 303, a reporter molecule 304, and a disulfide bond (—S—S—). 305 represents all other parts of a capture probe, for example a spatial barcode and a capture domain.

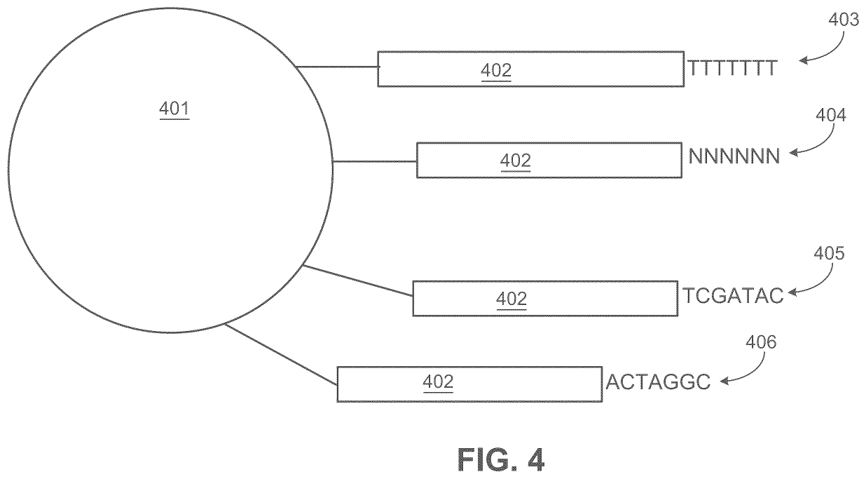

In some instances, the disclosure provides multiplexed spatially-barcoded features. FIG. 4 is a schematic diagram of an exemplary multiplexed spatially-barcoded feature. In FIG. 4, the feature 401 (e.g., a bead, a location on a slide or other substrate, a well on a slide or other substrate, a partition on a slide or other substrate, etc.) can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 402. One type of capture probe associated with the feature includes the spatial barcode 402 in combination with a poly(T) capture domain 403, designed to capture mRNA target analytes. A second type of capture probe associated with the feature includes the spatial barcode 402 in combination with a random N-mer capture domain 404 for gDNA analysis. A third type of capture probe associated with the feature includes the spatial barcode 402 in combination with a capture domain complementary to the analyte capture agent of interest 405. A fourth type of capture probe associated with the feature includes the spatial barcode 402 in combination with a capture probe that can specifically bind a nucleic acid molecule 406 that can function in a CRISPR assay (e.g., CRISPR/Cas9). While only four different capture probe-barcoded constructs are shown in FIG. 4, capture-probe barcoded constructs can be tailored for analyses of any given analyte associated with a nucleic acid and capable of binding with such a construct. For example, the schemes shown in FIG. 4 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

Additional features of capture probes are described in WO 2020/176788 and/or U.S. Patent Application Publication No. 2020/0277663, each of which is incorporated by reference in its entirety. Generation of capture probes can be achieved by any appropriate method, including those described in WO 2020/176788 and/or U.S. Patent Application Publication No. 2020/0277663, each of which is incorporated by reference in its entirety.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in WO 2020/176788 and/or U.S. Patent Application Publication No. 2020/0277663. Some quality control measures are described in WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in WO 2020/176788 and/or U.S. Patent Application Publication No. 2020/0277663. Exemplary features and geometric attributes of an array can be found in WO 2020/176788 and/or U.S. Patent Application Publication No. 2020/0277663.

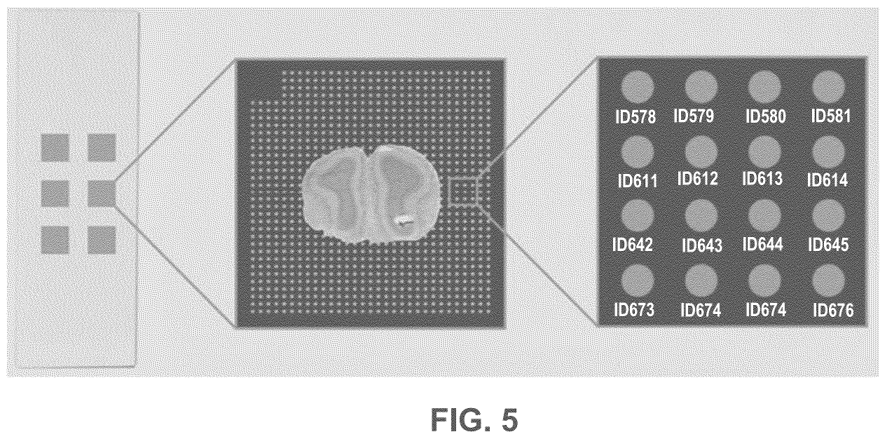

FIG. 5 depicts an exemplary arrangement of barcoded features on a substrate, in this case generating an array on a substrate. From left to right, FIG. 5 shows a slide including six spatially-barcoded arrays, an enlarged schematic of one of the six spatially-barcoded arrays, showing a grid of barcoded features in relation to a biological sample, and an enlarged schematic of one section of an array, showing the specific identification of multiple features within the array (labelled as ID578, ID579, ID560, etc.).

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells, areas on a substrate) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in WO 2020/176788 and/or U.S. Patent Application Publication No. 2020/0277663, or any of one or more of the devices or methods described in WO 2020/123320.

Suitable systems for performing spatial analysis can include components such as a chamber (e.g., a flow cell or sealable, fluid-tight chamber) for containing a biological sample. The biological sample can be for example, placed in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Selecting One or More Regions of Interest Using a Sealant

Spatial analysis workflows generally involve contacting a biological sample with a substrate, for example that comprises a plurality of barcoded capture probes. However, sometimes interrogating a whole tissue section is not needed or desired, and only a section or portion of a tissue section is of interest. In such instances, methods to select a region of interest for further evaluation are needed. Provided herein are methods for capturing an analyte from a first region of interest of a biological sample on a substrate, where the biological sample comprises the first region of interest and a second region (e.g., a region of non-interest), wherein the method includes contacting the second region with a sealant in order to create a hydrophobic seal, thereby preventing an interaction between an analyte with a capture domain of a capture probe at the second region. Although a first region and a second region are described in this application, it is appreciated that there can be multiple first regions of interest (i.e., multiple regions of interest) and multiple second regions (i.e., multiple regions of non-interest). In addition, in some instances, the second region can be identified as one or more areas of a substrate upon which no biological sample is placed.

The disclosure is predicated on the discovery that downstream analysis (e.g., sequencing) can result in increased reads or clearer understanding of analyte spatial analysis if regions on a substrate that are not of interest (e.g., second regions; regions where no biological sample is present or regions where a biological sample is present but is not the region of interest in the biological sample) do not capture analytes and do not contribute to downstream analysis.

In some instances, the methods include providing a biological sample on a substrate that includes a plurality of capture probes. In some instances, the plurality of capture probes include a capture domain and a spatial barcode. In some instances, the biological sample on the substrate is stained using any of the methods described herein. In some instances, the biological sample is imaged, capturing a staining pattern created during the stain step. In some instances, the biological sample is optionally destained. After optional destaining, in some instances, the biological sample is contacted with a sealant, wherein the sealant generates a hydrophobic seal that covers a region of the biological sample and prevents an interaction between an analyte from the region with a capture domain of the capture probe on the substrate. In some instances, the biological sample is permeabilized using methods disclosed herein (e.g., a solution that includes proteinase K and SDS). Permeabilization releases the analytes from the biological sample. Permeabilization can be performed contemporaneously with or after contacting the substrate with the biological sample. Analytes migrate from the biological sample and are captured by capture probes on the substrate. In some instances, after capture, the analytes and/or the probe can be amplified and the sequence can be determined using methods disclosed herein.

The method can further include imaging steps using any appropriate method described herein followed by a permeabilization step. In some embodiments, the imaging step comprises immunofluorescence or H&E stain. In some instances, the permeabilization step can include the use of pepsin and hydrochloric acid. Following the permeabilization step is standard cDNA synthesis and library preparation as further described herein.

Accordingly, provided herein are materials and methods that allow a user to create a hydrophobic seal of one or more regions of interest of the biological sample, for example, to prevent analyte capture of the analytes corresponding to the region of interest covered by the hydrophobic seal. In some embodiments, a hydrophobic seal is reversible.

Provided herein are methods for capturing an analyte from a first region of interest of a biological sample on a substrate, wherein the biological sample comprises the first region of interest and a second region, the method comprising: (a) contacting the biological sample with a substrate comprising a plurality of capture probes wherein a capture probe comprises a spatial barcode and the capture domain; (b) contacting the second region with a sealant, wherein the sealant generates a hydrophobic seal that covers the second region, thereby preventing an interaction between an analyte from the second region with a capture domain of a capture probe; and (c) hybridizing the analyte from the first region of interest to the capture probe, thereby capturing the analyte from the first region of interest of the biological sample.

Also provided herein are methods for capturing an analyte from a first region of interest of a biological sample, wherein the biological sample comprises the first region of interest and a second region, the method comprising: (a) contacting the biological sample with a substrate comprising a plurality of capture probes comprising the capture probe, wherein the capture probe comprises a spatial barcode and a capture domain; (b) providing a biological sample comprising: (i) the first region of interest, and (ii) the second region covered with a sealant, wherein the sealant generates a hydrophobic seal that covers the second region, thereby preventing an interaction between an analyte from the second region with a capture domain of a capture probe; and (c) hybridizing the analyte from the first region of interest to the capture probe, thereby capturing the analyte from the first region of interest of the biological sample. In some embodiments, the method further includes, prior to step (a), applying the sealant to the second region, such that the sealant forms a hydrophobic seal that covers the second region, thereby blocking the interaction between the analyte from the second region and the capture domain of the capture probe.

Finally, also provided herein are methods for capturing an analyte from a first region of interest of a biological sample, wherein the biological sample comprises the first region of interest and a second region, the method comprising: (a) providing a biological sample comprising: (i) the first region of interest, and (ii) the second region covered with a sealant, wherein the sealant generates a hydrophobic seal that covers the second region, thereby preventing an interaction between an analyte from the second region with a capture domain of a capture probe; (b) contacting the biological sample with a substrate comprising a plurality of capture probes comprising the capture probe, wherein the capture probe comprises a spatial barcode and a capture domain; (c) applying the sealant to the second region, such that the sealant forms a hydrophobic seal that covers the second region, thereby blocking the interaction between the analyte from the second region and the capture domain of the capture probe; and (d) hybridizing the analyte from the first region of interest to the capture probe, thereby capturing the analyte from the first region of interest of the biological sample.

In some embodiments of any of the methods described herein, the method further includes determining (i) all or a portion of a sequence corresponding to the analyte bound to the capture domain or a complement thereof, and (ii) the sequence corresponding to the spatial barcode or a complement thereof, and using the determined sequences of (i) and (ii) to determine the abundance or location of the analyte in the biological sample.

In some embodiments, a biological sample is placed on a substrate. In some embodiments, a biological sample is placed on the substrate prior to performing the methods described herein.

In some embodiments, a substrate can be used to provide support to a biological sample, particularly, for example, a thin tissue section. In some embodiments, mounting the biological sample onto the substrate includes sectioning of a tissue sample (e.g., cryostat sectioning) followed by a fixation step. In some instances, the fixation step can include fixation with methanol. In some instances, the fixation step includes formalin (e.g., 2% formalin).

Further, a “substrate” (e.g., a first substrate, a second substrate, a third substrate, or more substrates) as used herein, and when not preceded by the modifier “chemical”, refers to a member with at least one surface that generally functions to provide physical support for biological samples, analytes, and/or any of the other chemical and/or physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and systems described herein include, but are not limited to, slides (e.g., slides formed from various glasses, slides formed from various polymers), hydrogels, layers and/or films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings, as will be discussed in further detail below. A wide variety of different substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Accordingly, a “substrate” is a support that is insoluble in aqueous liquid and which allows for positioning of biological samples, analytes, features, and/or capture probes on the substrate. In many of the methods described herein, features (as described further below) are collectively positioned on a substrate. An “array” is a specific arrangement of a plurality of features that is either irregular or forms a regular pattern. Individual features in the array differ from one another based on their relative spatial locations. In general, at least two of the plurality of features in the array include a distinct capture probe (e.g., any of the examples of capture probes described herein).

In some embodiments, a workflow described herein comprises contacting a biological sample on a substrate with at least one feature array of the substrate.

### (a) Regions of Interest

In some embodiments, the biological sample includes a first region of interest. In some embodiments, a region of interest includes the entire biological sample. In some embodiments, a first region of interest includes a portion of the biological sample. In some embodiments, a biological sample also includes a second region, which is typically not of interest in the sense that a researcher is not interested in spatially assaying the second region. It is understood that a “first” region and a “second” region are designators used for convenience, and any two areas may be used to designate a region of interest and a region of non-interest. Further in some instances, more than one region of interest is identified in a biological sample. For instance, a biological sample can include at least one, two, three, four, or more regions of interest and so on. In some instances, the substrate can include multiple regions (e.g., at least one, two, three, four, or more) that are identified as “not of interest”. As such, a biological sample could include one or more regions of interest where determining spatial information is desirous, and one or more other regions where determination of spatial information is not desirous.

In some embodiments, a first region of interest can be confined to a first portion of a biological sample, and a second region can be confined to a second portion of the biological sample.

In some embodiments, a region of interest (e.g., a first region of interest) corresponds to an anatomical feature within the biological sample. For instances, a specific type of cell can comprise a region of interest. In some instances, a specific pathology (e.g., a region identified as containing cancer cells) comprises the region of interest (e.g., a first region of interest).

In some embodiments, a region of interest (e.g., a first region of interest) corresponds to coordinates on a substrate. In such cases, the coordinates could be identified manually (e.g., visual inspection) or using a trained tissue detection machine learning module.

In some embodiments of any of the methods described herein, a biological sample can include a plurality of regions of interest (e.g., multiple first regions of interest). In such cases, one or more regions that are not of interest can be contacted with a sealant, wherein the sealant creates a hydrophobic seal covering the one or more regions not of interest (e.g., a second region) thereby preventing interaction between an analyte corresponding to the one or more regions not of interest and a capture probe on a substrate. As described herein, the hydrophobic seal covering the one or more regions not of interest can be removed using any of the methods described herein.

### (b) Identifying One or More Regions of Interest

In some embodiments, the methods described herein include identifying one or more regions of interest (e.g., the first region of interest; multiple first regions of interest). In some embodiments, the method includes identifying a first region of interest and not identifying a second region. In such cases where a first region of interest is identified, the remaining biological sample on the substrate can be considered the second region that is not of interest. For example, where the method includes identifying a first region of interest but not a second region, the biological sample that does not correspond to the first region of interest can be considered the second region. In some cases, identifying the first region of interest and the second region is performed prior to contacting the biological sample with a sealant. Non-limiting examples of methods of identifying the first region of interest and the second region that may or may not be of interest include identifying manually (e.g., visual inspection) or using a tissue detection machine learning module, for example HALO AI (Indicia Labs) and ONCOTOPIX (Visiopharm), and as described in Tomita et al. (JAMA Network Open. 2019, 2(11)e1914645), Bychkov et al. (Scientific Reports, 2018, 8:3395), and Tsai and Tao, Electronics 2021, 10, 1662, each of which is incorporated by reference in its entirety. In some embodiments, identifying a first region of interest and a second region include visual inspection following staining.

In some embodiments, the trained machine learning module includes at least one of a supervised learning module, a semi supervised learning module, an unsupervised learning module, a regression analysis module, a reinforcement learning module, a self-learning module, a feature learning module, a sparse dictionary learning module, an anomaly detection module, a generative adversarial network, a convolutional neural network, or an association rules module. For example, the first region of interest and the second region are identified by a supervised machine learning module. In another example, the first region of interest and the second region are identified by a tissue detection machine learning module.

In some embodiments, identifying a first region of interest and/or a second region include detecting a signal corresponding to one or more analytes of interest. In such cases, the signal corresponding to the one or more analytes in a first region of interest can include a signal from a conjugated antibody bound to the one or more analytes, conjugated secondary antibody bound to a primary antibody bound to the one or more analyte, a labelled nucleotide, a labelled oligonucleotide, a labelled oligonucleotide probe, or any combination thereof. In some embodiments, the one or more analytes of interest include, without limitation, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, extracellular and intracellular proteins, antibodies, and antigen binding fragments. In some embodiments, the signal corresponding to the one or more analytes identifies a first region of interest.

### (c) Sealant

In some embodiments, the methods provided herein include a sealant to the biological sample. In some embodiments, a sealant is capable of generating a hydrophobic seal. As used herein, the term “hydrophobic seal” refers to a seal that is impervious to water. In some cases, the region of interest of the biological sample that is contacted with the sealant can be referred to as being “sealed.” In some embodiments, the biological sample is contacted with the sealant under conditions such that a hydrophobic seal is generated after a period of time.

In some instances, the sealant is an organic adhesive. In some instances, the sealant includes d-limonene, which is a natural solvent that dries to form a clear, hard, durable seal. In some instances, the sealant forms a clear, hard seal on the capture probes.

In some instances, the sealant includes one or more polymers and/or one or more polymer resins. In some instances, the polymer is nitrocellulose. In some instances, the sealant is dissolved in a solvent, including but not limited to ethyl acetate or butyl acetate.

In some embodiments, the sealant is selected from the group consisting of: a coverslip sealant, a liquid coverslip, a mounting media, a gel, an adhesive, and a bilayer, or a combination thereof. In some embodiments, the sealant comprises a coverslip sealant. In some embodiments, the coverslip sealant is Covergrip™ Coverslip Sealant (Biotium). In some embodiments, the sealant is a commercially available nail polish, such as a clear nail polish (e.g., Sally Hansen brand nail polish or any other commercially available brand).

In some instances, the sealant is formed using the solution from a hydrophobic (PAP) pen traditionally used to draw a hydrophobic circle around the tissue in the setting of immunostaining. In some instance, the solution from a hydrophobic pen is insoluble in ethanol, acetone and water.

In some embodiments, contacting the biological sample with the substrate occurs prior to or contemporaneously with the time when the sealant has formed a hydrophobic seal. In some embodiments, contacting the biological sample with the substrate occurs before the sealant has formed a hydrophobic seal.

In some embodiments, the period of time for the sealant to form a hydrophobic seal includes about 15 minutes to about 4 hours (e.g., about 15 minutes to about 3.5 hours, about 15 minutes to about 3 hours, about 15 minutes to about 2.5 hours, about 15 minutes to about 2 hours, about 15 minutes to about 1.5 hours, about 15 minutes to about 1.0 hour, about 15 minutes to about 45 minutes, about 15 minutes to about 30 minutes, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1.0 hour, about 30 minutes to about 45 minutes, about 45 minutes to about 4 hours, about 45 minutes to about 3.5 hours, about 45 minutes to about 3 hours, about 45 minutes to about 2.5 hours, about 45 minutes to about 2 hours, about 45 minutes to about 1.5 hours, about 45 minutes to about 1.0 hour, about 1.0 hour to about 4 hours, about 1.0 hour to about 3.5 hours, about 1.0 hour to about 3 hours, about 1 hour to about 2.5 hours, about 1 hour to about 2 hours, about 1 hour to about 1.5 hours, about 1.5 hours to about 4 hours, about 1.5 hours to about 3.5 hours, about 1.5 hours to about 3 hours, about 1.5 hours to about 2.5 hours, about 1.5 hours to about 2 hours, about 2 hours to about 4 hours, about 2.0 hours to about 3.5 hours, about 2.0 hours to about 3 hours, about 2.0 hours to about 2.5 hours, about 2.5 hours to about 4 hours, about 2.5 hours to about 3.5 hours, about 2.5 hours to about 3.0 hour, about 3.0 hours to about 4 hours, about 3.0 hours to about 3.5 hours, or about 3.5 hours to about 4 hours). In some embodiments, the period of time for the sealant to form a hydrophobic seal is about 1.0 hours.

In some embodiments, the methods provided herein include contacting the biological sample with a sealant. In some embodiments, contacting the biological sample with a sealant includes applying the sealant using a method selected from: a brush tip, a dropper, a pipette, a microfluidic device, and a liquid handling instrument. The sealant can be applied onto the biological sample. In some embodiments, the applying the sealant comprises automation.

In some embodiments, applying the sealant includes identifying the first region of interest from the second region; and applying the sealant using automation to the second region. In some embodiments, applying the sealant includes identifying multiple first regions of interest and multiple second regions; and applying the sealant using automation to the multiple second regions.

In some embodiments, the automation comprises a system comprising a computer implemented method and a liquid handling instrument. In some embodiments, a computer implemented method can be used to train a machine learning module (e.g., a tissue detection machine learning module) and determine, using the machine learning module, a first region of interest and a second region. In such cases, a computer implemented method includes: generating a dataset of a plurality of biological samples (e.g., one or more reference samples), wherein the dataset comprises, for each biological sample of the plurality of biological samples: (i) analyte data for a plurality of analytes at a plurality of spatial locations of a reference biological sample; (ii) image data of the reference biological sample; and (iii) registration data of the imaged data linking to the analyte data according to the spatial locations of the reference biological sample; wherein the reference biological sample comprises (1) a first region of interest in the reference biological sample, and (2) a second region that may not be of particular interest; (b) training a machine learning module with the dataset, thereby generating a trained tissue detection machine learning module; and (c) identifying a first region of interest and/or a second region in a biological sample via the trained machine learning module.

### (d) Permeabilization

In some embodiments, the method also includes contacting the biological sample with a permeabilization agent, wherein the permeabilization agent is selected from an organic solvent, a detergent, and an enzyme, or a combination thereof. Non-limiting examples of permeabilization agent include without limitation: an endopeptidase, a protease sodium dodecyl sulfate (SDS), polyethylene glycol tert-octylphenyl ether, polysorbate 80, and polysorbate 20, N-lauroylsarcosine sodium salt solution, saponin, Triton X-100™, and Tween-20™.

In some embodiments, the permeabilizing step is performed after contacting the biological sample with the substrate. In some embodiments, the permeabilizing step is performed after the sealant is allowed to form a hydrophobic seal.

In some embodiments, permeabilization occurs using a protease. In some embodiments, the protease is an endopeptidase. In some embodiments, the endopeptidase is pepsin or proteinase K. Other endopeptidases that can be used include but are not limited to trypsin, chymotrypsin, elastase, thermolysin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, after creating a seal on the second region, the biological sample is permeabilized.

In some embodiments, methods provided herein include permeabilization of the biological sample such that the capture probe is more readily accessible for hybridizing to the analyte (i.e., compared to no permeabilization). In some embodiments, reverse transcription (RT) reagents can be added to permeabilized biological samples. Incubation with the RT reagents can produce sequences complementary to the analyte that is hybridized to the capture probe. In some embodiments, second strand reagents (e.g., second strand primers, enzymes, labeled and unlabeled dNTPs) can be added to the biological sample on the slide to initiate second strand synthesis.

In some instances, the permeabilization step includes application of a permeabilization buffer to the biological sample. In some instances, the permeabilization buffer includes a buffer (e.g., Tris pH 7.5), MgCl₂, sarkosyl detergent (e.g., sodium lauroyl sarcosinate), enzyme (e.g., proteinase K), and nuclease free water.

In some instances, the permeabilization buffer includes a ribonuclease inhibitor. In some instances, the ribonuclease inhibitor includes ribonucleoside vanadyl complex (RVC). In some instances, the permeabilization buffer includes a ribonuclease inhibitor and a reducing agent. For example, the permeabilization buffer includes RVC and DTT. In some instances, RVC is added to the permeabilization buffer at a final concentration of about 2 mM to about 20 mM (e.g., about 2 mM to about 15 mM, about 2 mM to about 10 mM, about 2 mM to about 5 mM, about 5 mM to about 20 mM, about 5 mM to about 15 mM, about 5 mM to about 10 mM, about 10 mM to about 20 mM, about 10 mM to about 15 mM, or about 15 mM to about 20 mM). In some instances, RVC is added to the permeabilization at a final concentration of about 10 mM.

In some instances, the permeabilization step is performed at 37° C. In some instances, the permeabilization step is performed for about 5 minutes to 2 hours (e.g., about 5 minutes, 10 minutes, about 20 minutes, about 30 minutes, about 40 minutes, about 50 minutes, about 1 hour, about 1.5 hours, or about 2 hours). In some instances, the permeabilization step is performed for about 40 minutes. In some embodiments, the permeabilization parameters are varied such that the optimal permeabilization conditions for a particular tissue type or sample can be determined for optimal tissue nucleic acid capture by the capture probe on the substrate.

### (e) Removing the Hydrophobic Seal

Provided herein are methods where the sealant is removed from the biological sample and the analytes corresponding to the region that was sealed prior to removal of the sealant can be analyzed (e.g., the sequence of the analyte (e.g., mRNA) can be determined). In such cases, the method includes removing the sealant from the biological samples (e.g., the second region); contacting the biological sample with a plurality of capture probes, where a capture probe of the plurality of capture probes comprises a spatial barcode and a capture domain, thereby allowing the analyte from the second region to bind to the capture domain of the capture probe from the substrate. In some instances, the sequence of the analytes from the second region (i.e., the analytes corresponding to region from which the sealant was removed) can be determined. In such cases, the method further includes determining (i) all or a portion of a sequence corresponding to the analyte from the second region specifically bound to the capture domain of a capture probe of the substrate or a complement thereof, and (ii) the sequence corresponding to the spatial barcode of the capture probe of the substrate or a complement thereof, and using the determined sequences of (i) and (ii) to determine the abundance or location of the analyte from the second region in the biological sample.

In some embodiments, removing the sealant from the biological sample includes, without limitation, lifting, peeling, dissolving, liquefying, or decrosslinking the sealant from the biological sample. In some embodiments, removing the sealant from the biological sample includes contacting the biological sample with a removing agent selected from the group consisting of: a solvent, an acid, a base, and a buffer, or any combinations thereof. In some embodiments, the removing agent comprises phosphate buffered saline (PBS). In some embodiments, removing comprises contacting the sealant with PBS.

In some embodiments, the method includes contacting the sealant with a removing agent for about 15 minutes to about 4 hours (e.g., about 15 minutes to about 3.5 hours, about 15 minutes to about 3 hours, about 15 minutes to about 2.5 hours, about 15 minutes to about 2 hours, about 15 minutes to about 1.5 hours, about 15 minutes to about 1.0 hour, about 15 minutes to about 45 minutes, about 15 minutes to about 30 minutes, about 30 minutes to about 4 hours, about 30 minutes to about 3.5 hours, about 30 minutes to about 3 hours, about 30 minutes to about 2.5 hours, about 30 minutes to about 2 hours, about 30 minutes to about 1.5 hours, about 30 minutes to about 1.0 hour, about 30 minutes to about 45 minutes, about 45 minutes to about 4 hours, about 45 minutes to about 3.5 hours, about 45 minutes to about 3 hours, about 45 minutes to about 2.5 hours, about 45 minutes to about 2 hours, about 45 minutes to about 1.5 hours, about 45 minutes to about 1.0 hour, about 1.0 hour to about 4 hours, about 1.0 hour to about 3.5 hours, about 1.0 hour to about 3 hours, about 1 hour to about 2.5 hours, about 1 hour to about 2 hours, about 1 hour to about 1.5 hours, about 1.5 hours to about 4 hours, about 1.5 hours to about 3.5 hours, about 1.5 hours to about 3 hours, about 1.5 hours to about 2.5 hours, about 1.5 hours to about 2 hours, about 2 hours to about 4 hours, about 2.0 hours to about 3.5 hours, about 2.0 hours to about 3 hours, about 2.0 hours to about 2.5 hours, about 2.5 hours to about 4 hours, about 2.5 hours to about 3.5 hours, about 2.5 hours to about 3.0 hour, about 3.0 hours to about 4 hours, about 3.0 hours to about 3.5 hours, or about 3.5 hours to about 4 hours). In some embodiments, the methods include contacting the sealant with a removing agent for about 1.0 hours.

After removal, steps of permeabilizing the biological sample in the second regions and capturing analytes in the second region are performed using the permeabilizing and capturing methods disclosed herein.

### (f) Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. In some embodiments, where the tissue sample is the FFPE tissue sample, and the tissue sample is decrosslinked.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed. Staining the sample also allows one to determine regions of interest. For instance, after an H&E stain or protein detection (e.g., IF or IHC) stain, one can detect regions of the sample having increased immune infiltrates or having tumor cells. Then, one can add a sealant to regions that are not of interest.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

In some embodiments, the biological sample was previously stained. In some embodiments, the biological sample was previously stained using immunofluorescence or immunohistochemistry. In some embodiments, the biological sample was previously stained using hematoxylin and eosin.

### (g) Determining the Sequence of an Analyte

After an analyte from the biological sample has hybridized or otherwise been associated with a capture probe according to any of the methods described above in connection with the general spatial cell-based analytical methodology, the barcoded constructs that result from hybridization/association are analyzed. In some embodiments, the barcoded constructs include an analyte from the first region of interest. In some embodiments, the barcoded constructs include an analyte from the first region of interest, an analyte from the second region, or combinations thereof.

In some embodiments, the methods provided herein include determining, from one or more first regions of interest, (i) all or a portion of a sequence corresponding to the analyte bound to the capture domain or a complement thereof, and (ii) the spatial barcode or a complement thereof, and using the determined sequences of (i) and (ii) to determine the abundance or location of the analyte in the first region of interest in the biological sample.

In some embodiments, the methods provided herein include determining, from a second region, (i) all or a portion of a sequence corresponding to the analyte bound to the capture domain or a complement thereof, and (ii) the spatial barcode or a complement thereof, and using the determined sequences of (i) and (ii) to determine the abundance or location of the analyte in the second region biological sample.

In some embodiments, the methods include determining all or a portion of a sequence from a second region wherein the second region includes a hydrophobic seal. In such cases, the methods includes: removing the sealant from the second region; contacting the biological sample with a substrate comprising a plurality of capture probes, wherein a capture probe of the plurality of capture probes from the substrate comprises a spatial barcode and a capture domain, thereby allowing the analyte from the second region to bind to the capture domain of the capture probe from the substrate. This method also includes determining (i) all or a portion of a sequence corresponding to the analyte from the second region specifically bound to the capture domain of a capture probe of the substrate or a complement thereof, and (ii) the spatial barcode of the capture probe of the substrate or a complement thereof, and using the determined sequences of (i) and (ii) to determine the abundance or location of the analyte from the second region in the biological sample.

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the ligated probe and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze barcoded analyte (e.g., the ligation product). In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

Provided herein are methods that enable greater sequencing depth for a biological sample wherein at least a portion of the biological sample is contacted with a sealant. For example, the methods result in greater capture efficiency and potentially greater sequencing depth for the biological sample as compared to the capture efficiency and subsequent depth of sequencing for a biological sample not contacted with a sealant.

Some embodiments of these methods result in about a 1% increase to about a 100% increase (e.g., about a 1% increase to about a 90% increase, about a 10% increase to about a 80% increase, about a 20% increase to about a 70% increase, about a 30% increase to about a 60% increase, or about a 40% increase to about a 50% increase) in capture efficiency (e.g., as compared to capture efficiency for a biological sample not contacted with a sealant).

Provided herein are methods that result in a greater number of reads per spot for the biological sample as compared to the number of read per spot for a biological sample not contacted with a sealant.

Some embodiments of these methods result in about a 1% increase to about a 100% increase (e.g., about a 1% increase to about a 90% increase, about a 10% increase to about a 80% increase, about a 20% increase to about a 70% increase, about a 30% increase to about a 60% increase, or about a 40% increase to about a 50% increase) in the number of reads per spot (e.g., as compared to the number of reads per spot for a biological sample not contacted with a sealant).

Provided herein are methods that results in a greater number of unique molecule identifier (UMI) counts per spot for the biological sample as compared to the number of UMI counts per spot for a biological sample not contacted with a sealant.

Some embodiments of these methods result in about a 1% increase to about a 100% increase (e.g., about a 1% increase to about a 90% increase, about a 10% increase to about a 80% increase, about a 20% increase to about a 70% increase, about a 30% increase to about a 60% increase, or about a 40% increase to about a 50% increase) in the number of UMI counts per spot for the biological samples (e.g., as compared to the number of UMI counts per spot for a biological sample not contacted with a sealant).

### (h) Kits

Also provided herein are kits that can be used to perform any of the methods described herein. In some embodiments, a kit includes: (a) a sealant; (b) a substrate comprising a plurality of capture probes, wherein the capture probes comprises a spatial barcode and a capture domain; and (c) instructions for performing any of the methods described herein.

Another non-limiting example of a kit used to perform any of the methods described herein includes: (a) a sealant; (b) a substrate comprising a plurality of capture probes, wherein the capture probes comprises a spatial barcode and a capture domain; (c) a removing agent; and (d) instructions for performing any of the methods described herein.

In some embodiments of any of the kits described herein, the sealant comprises a coverslip sealant. In some embodiments of any of the kits described herein, the coverslip sealant is Covergrip™ Coverslip Sealant (Biotium). In some embodiments, the sealant is nail polish. In some embodiments of any of the kits described herein, the kit also includes a sealant applicator. In some embodiments of any of the kits described herein, the kit also includes a removing agent. In some embodiments of any of the kits described herein, the removing agent is PBS.

### EXAMPLES

### Example 1. Spatial Analysis of a Region of Interest using a Sealant

This example provides an exemplary method for analyzing an analyte in a biological sample from a region of interest. The example demonstrates that using a sealant over an arca of a biological sample results in an increased number of reads in areas where no sealant was used on that biological sample, relative to a sample where no sealant was used on any area of the biological sample. Thus, by using a sealant, one can generate increased reads in a region of interest (e.g., in a region without a sealant).

The sample was preserved by FFPE processing. Before addition of the sealant, the biological samples were deparaffinized and stained per established protocols. For example, FFPE tissue samples were prewarmed in a water bath (40° C.), sectioned (10 μm), dried at 42° C. for several hours and placed in a desiccator at room temperature overnight. The dry, sectioned tissues were deparaffinized by baking at 60° C., moved through a series of xylene and EtOH washes, rinsed in water several times. Following rinsing, the deparaffinized tissues were stained with hematoxylin per established protocols. The stained tissues were imaged.

The tissues were decrosslinked to remove formaldehyde crosslinks within the sample thereby making the analytes accessible for capture. Briefly, the tissue samples were incubated with an HCl solution for 1 minute, repeated twice for a total of 3 minutes. Following HCl incubations, the tissue sections were incubated at 70° C. for 1 hour in TE pH 9.0. TE was removed and the tissues were incubation in 1×PBS-Tween for 15 minutes.

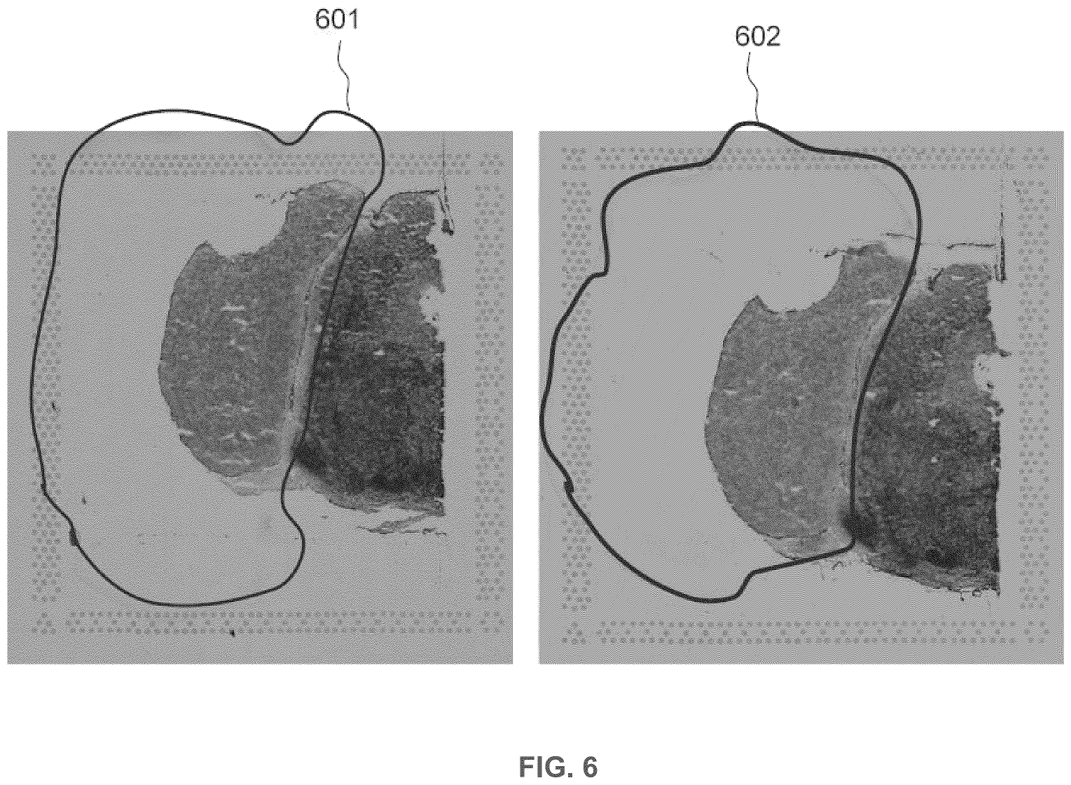

After contacting the biological sample located on the substrate that includes capture probes, a region where the analytes are prevented from interacting with a capture probe was contacted with a sealant (e.g., Covergrip™ Coverslip Sealant) (see FIG. 6). The black lines 601 and 602 surround the region in each biological sample to which the sealant was applied. The region not covered by a sealant (i.e., the right side of each image) is considered the first region of interest, and the region with the sealant is considered a second region. The sealant dried for one hour at room temperature in order to form a hydrophobic seal over the second region.

The biological sample was permeabilized to release the analytes from the region of interest. In particular, after 30 minutes, the tissues were washed and permeabilized by adding either Proteinase K or Pepsin, incubated at 37° C. for at least 5 minutes and washed to remove the protease.

The analytes from the region of interest hybridized to the capture probes. The sealant prevented permeabilization of the second region, thereby blocking the release of the analytes. It is appreciated that the sealant also directly prevented the release of the analytes from the second region in addition to preventing permeabilization.

The analytes from the region of interest (e.g., the first region of interest not covered by the sealant) that hybridized to the capture probes were used as a template in a nucleic acid extension reaction that generated extended capture probes. The extended capture probes were amplified and sequenced according to any one of the methods described herein. Subsequent sequence analysis was used to determine spatial information regarding the analyte captured from the tissue sample.

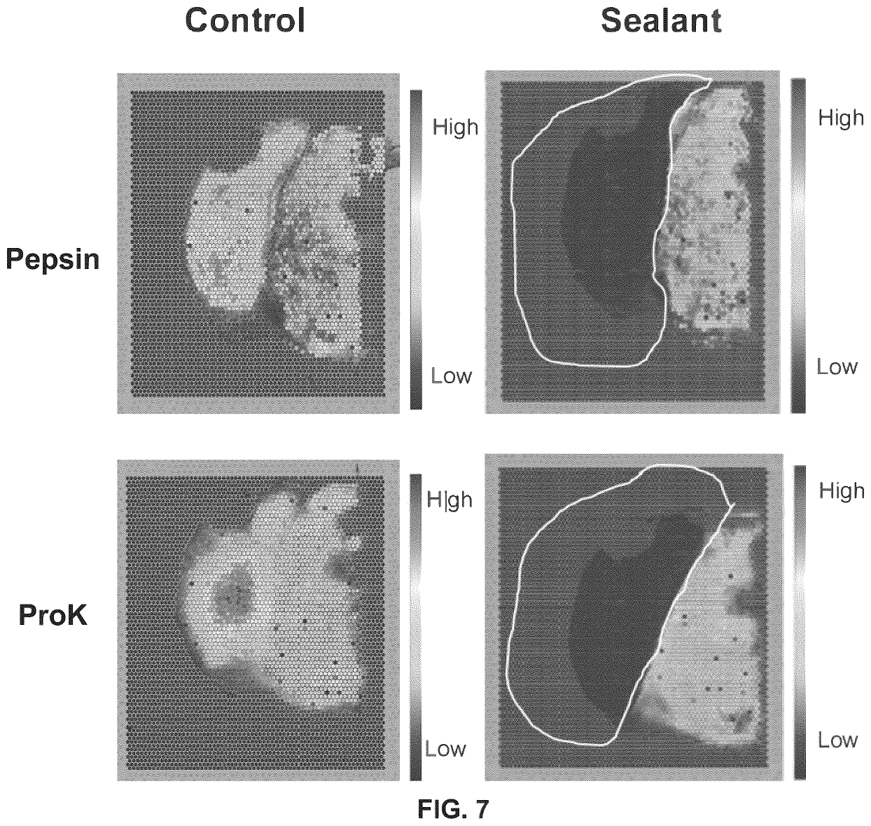

As shown in the gene expression heat maps in FIG. 7, the sealant prevented interaction between analytes from the second region with capture domains of the capture probe. See FIG. 7, right “Sealant” images. However, without a sealant (FIG. 7, left “Control” images), analytes were captured from the entire sample. Further, both pepsin and proteinase K were used to permeabilize the sample, and based on the images in FIG. 7, each permeabilization reagent (1) detected analytes in the first region of interest, and (2) did not permeabilize the sample in the second region. This was evident by the very low UMI counts (0-500 counts per spot). In this experiment, a serial section of the same tissue sample was used as control.

Table 1 reports the outcome of the experiments comparing conditions on control tissue (e.g., the whole tissue was assayed) versus tissues where a sealant was applied on a region of the tissue (e.g., a region of interest, and not the whole tissue, was assayed). Comparative results demonstrate the increase in resolution of the regions of interest from the tissues where a portion of the tissue was sealed versus the whole tissue where there was no sealant.

TABLE 1
Sensitivity comparison between controls and sealant samples.

| | | | | | Median UMI |
|---|---|---|---|---|---|
| | | Median | Median genes | Median UMI | counts per spot |
| | | genes per | per spot (50 k | counts per | (50 k mapped |
| | | spot (50 k | mapped spot- | spot (50 k | spot-reads per |
| Condition | Perm | rrps) | reads per spot) | rrps) | spot) |
| sealant | Pepsin | 5570 | 5998 | 23152 | 27057 |
| control | Pepsin | 4269 | 5833 | 14208 | 25803 |
| sealant | ProK | 6171 | 6601 | 30455 | 35484 |
| control | ProK | 4911 | 6478 | 19216 | 34658 |

Table 2 shows sequencing metrics for a tissue region of interest (e.g., a portion of the tissue was sealed with a sealant) compared to a whole tissue (e.g., the whole tissue was assayed). The results of these experiments report a higher sequencing saturation for sealant samples as compared to control samples when matched for spots corresponding to tissues where a sealant was applied and a region of interest was assayed versus a tissue where there was no sealant applied and the whole tissue was assayed.

TABLE 2
Sequencing statistics for the control and sealant samples.

| Condition | sealant | control | sealant | control |
|---|---|---|---|---|
| Perm | pepsin | pepsin | proK | proK |
| No. spots covered by | 1241 | 1245 | 1209 | 1254 |
| tissue | | | | |
| Sequencing saturation | 0.72 | 0.57 | 0.62 | 0.43 |
| Mean reads/spot | 199243 | 188710 | 187008 | 165290 |
| Valid barcodes | 98% | 98% | 98% | 98% |
| Reads mapped | 85% | 84% | 88% | 87% |
| confidently to | | | | |
| transcriptome | | | | |
| Fraction reads in spot | 93% | 47% | 93% | 53% |
| Median gene/spot (50 k | 5570 | 4269 | 6171 | 4911 |
| rrps) | | | | |
| Median gene/spot (50 k | 5998 | 5833 | 6601 | 6478 |
| mapped spot-reads/spot) | | | | |
| Median UMI/spot (50 k | 23152 | 14208 | 30455 | 19216 |
| rrps) | | | | |
| Median UMI/spot (50 k | 27057 | 25803 | 35484 | 34658 |
| mapped spot-reads/spot) | | | | |

Collectively, the results demonstrate the sealant does not have a negative effect on workflow and actually results in greater number of reads per spot, greater fraction of library complexity being captured during sequencing and greater number of unique molecular identifiers (UMIs) per spot for the region of interest not covered by a sealant as compared a biological sample where a whole tissue was assayed and not just a region of interest.

### Example 2. Spatial Analysis of Spleen and Liver Biological Samples using a Sealant

This example provides an exemplary method for analyzing an analyte in a biological sample (i.e., spleen and liver samples) at a region of interest where regions not of interest are sealed off using a sealant.

Liver and spleen samples were preserved by FFPE processing. Before addition of the sealant, the biological samples were deparaffinized and stained per established protocols. For example, FFPE tissue samples were prewarmed in a water bath (40° C.), sectioned (10 μm), dried at 42° C. for several hours, and placed in a desiccator at room temperature overnight. The dry, sectioned tissues were deparaffinized by baking at 60° C., moved through a series of xylene and EtOH washes, rinsed in water several times. Following rinsing, the deparaffinized tissues were stained with hematoxylin per established protocols. The stained tissues were imaged.

The tissues were decrosslinked to remove formaldehyde crosslinks within the sample thereby making the analytes accessible for capture. Briefly, the tissue samples were incubated with an HCl solution for 1 minute, repeated twice for a total of 3 minutes. Following HCl incubations, the tissue sections were incubated at 70° C. for 1 hour in TE pH 9.0. TE was removed and the tissues were incubation in 1×PBS-Tween for 15 minutes.

An area on a substrate was identified as a region of interest. The area outside of the region of interest was covered with a sealant (i.e., COVERGRIP™ sealant or SALLY HANSEN™ BRAND nail polish). The COVERGRIP™ sealant or SALLY HANSEN™ nail polish were allowed to dry at room temperature for 30 minutes. The area outside of the region of interest can include part of the biological sample or no biological sample.

The liver and spleen tissue sections were permeabilized to release the analytes from the region of interest. In particular, after 30 minutes, the tissues were washed and permeabilized by adding either proteinase K or pepsin, incubated at 37° C. for at least 5 minutes and then washed to remove the proteinase K or pepsin.

The analytes from the biological sample(s) hybridized to the capture probes. The sealant prevented permeabilization of the area outside of the region of interest, thereby blocking the release of the analytes in that sealed region. The sealant also prevented the release of the analytes from the area outside of the region of interest in addition to preventing permeabilization.

The analytes from the biological sample(s) that hybridized to the capture probes were used as a template in a nucleic acid extension reaction that generated extended capture probes. The extended capture probes were amplified and sequenced according to any one of the methods described herein. Subsequent sequence analysis was used to determine spatial information regarding the analyte captured from the tissue sample region of interest. Control samples were also assayed, except there was no sealant applied to any part of the control tissue or slide.

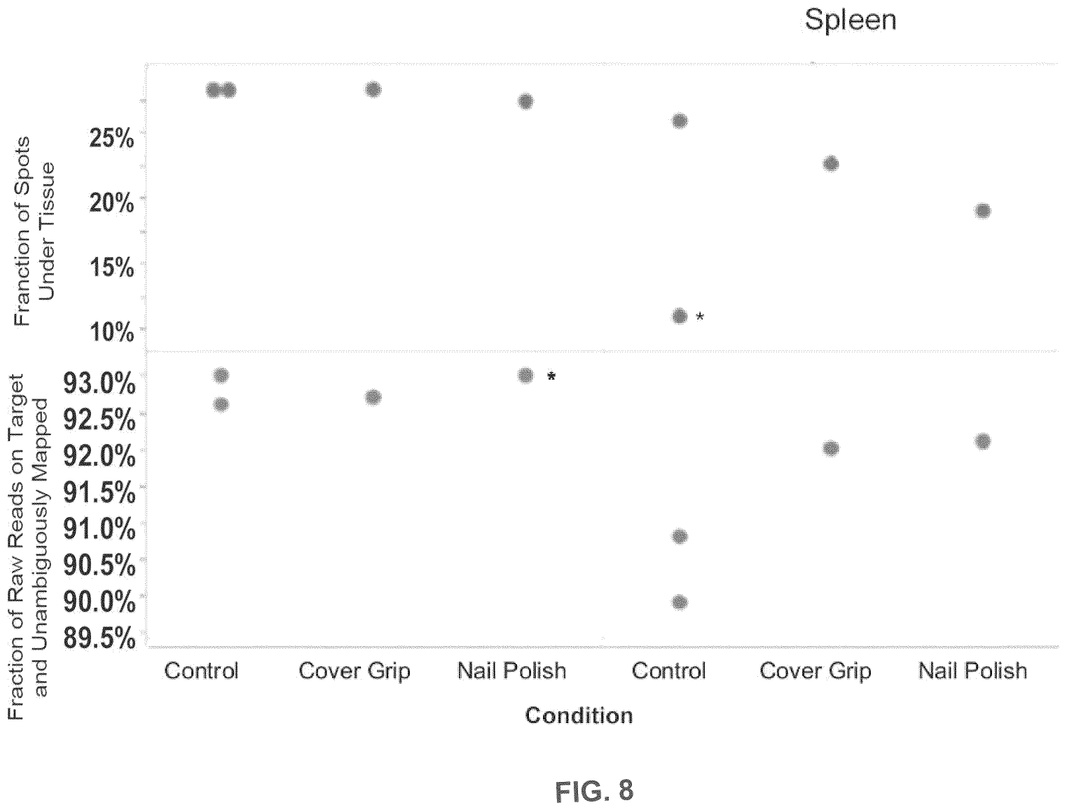

Further comparisons were made between biological samples (e.g., tissue samples) that included the sealant covering an area outside of the biological sample and controls where no sealant was applied. FIG. 8 shows fraction of raw reads on target and unambiguously mapped (bottom panels), and fractions of spots under tissue (top panels) of liver (left panels) and spleen (right panels) tissue samples that were positioned on the substrate where an area outside and around the tissue sample (1) had no sealant applied and was unblocked (control), (2) an area around the tissue sample was blocked with cover grip (e.g., COVERGRIP™) sealant, and or (3) an area around the tissue sample was blocked with nail polish (e.g., SALLY HANSEN™). Each spot is representative of a where an area outside of the tissue sample (1) had no sealant applied and was unblocked (control), (2) an area around the tissue sample was blocked with cover grip (e.g., COVERGRIP™) sealant, or (3) an area around the tissue sample was blocked with nail polish (e.g., SALLY HANSEN™). FIG. 8 shows the fraction of raw reads on target and unambiguously mapping rate is slightly higher when the area outside of the tissue sample is blocked by the application of a sealant. The spot (bottom panel) marked with an asterisk indicates a liver sample where the nail polish sealant failed (e.g., came off the substrate). The right spot marked with asterisk (top panel) is a control spleen sample where the spleen moved during the assay to an area that was substantially off of the substrate.

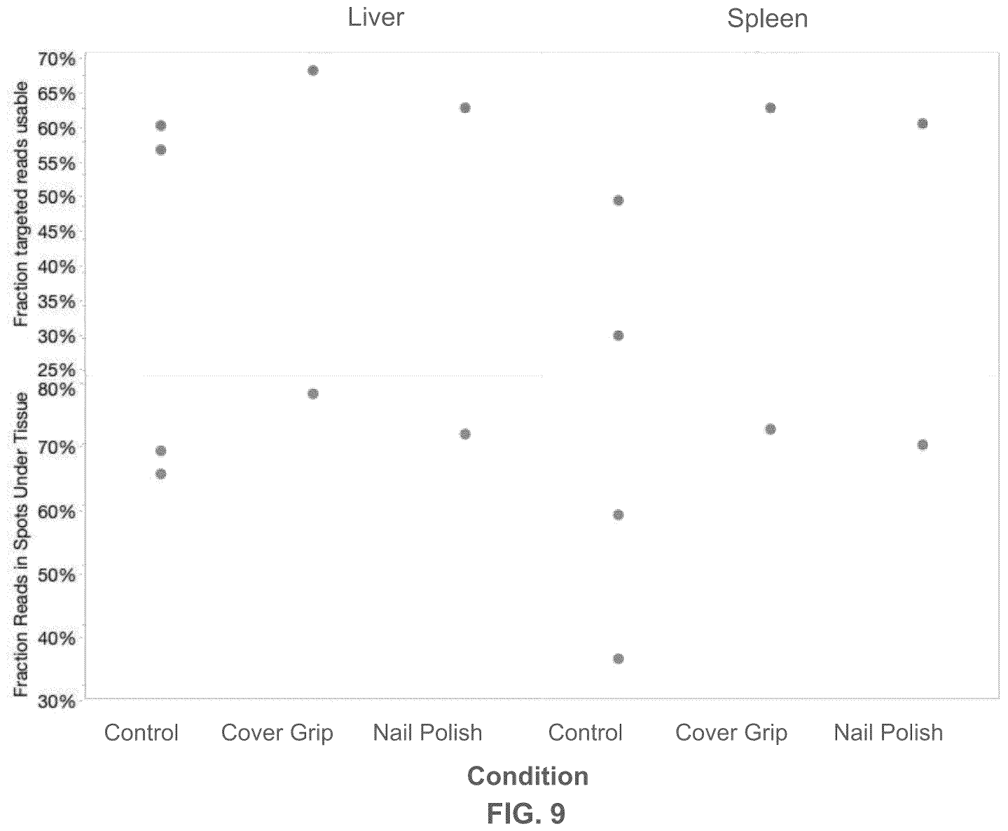

FIG. 9 shows the fraction of reads in spots under the tissue (bottom panels), and fraction of targeted reads useable (top panels) of liver (left panels) and spleen (right panels) tissue samples were positioned on a substrate where an area outside of and around the tissue sample (1) had no sealant applied and was unblocked (control), (2) an area around the tissue sample was blocked with cover grip (e.g., COVERGRIP™) sealant, or (3) an area around the tissue sample was blocked with nail polish (e.g., SALLY HANSEN™). FIG. 9 shows an increase in fraction of target reads, particularly in the spleen samples, when the area outside of the tissue sample is blocked thus preventing interaction between a capture domain of a capture probe and an analyte from the tissue sample. The fraction of target reads in spots under the tissue substantially match the fraction of targeted reads usable for each of the samples and sample conditions. Fraction of reads in about 10% greater for the tissue sample when the area around the tissue sample is blocked with a sealant.

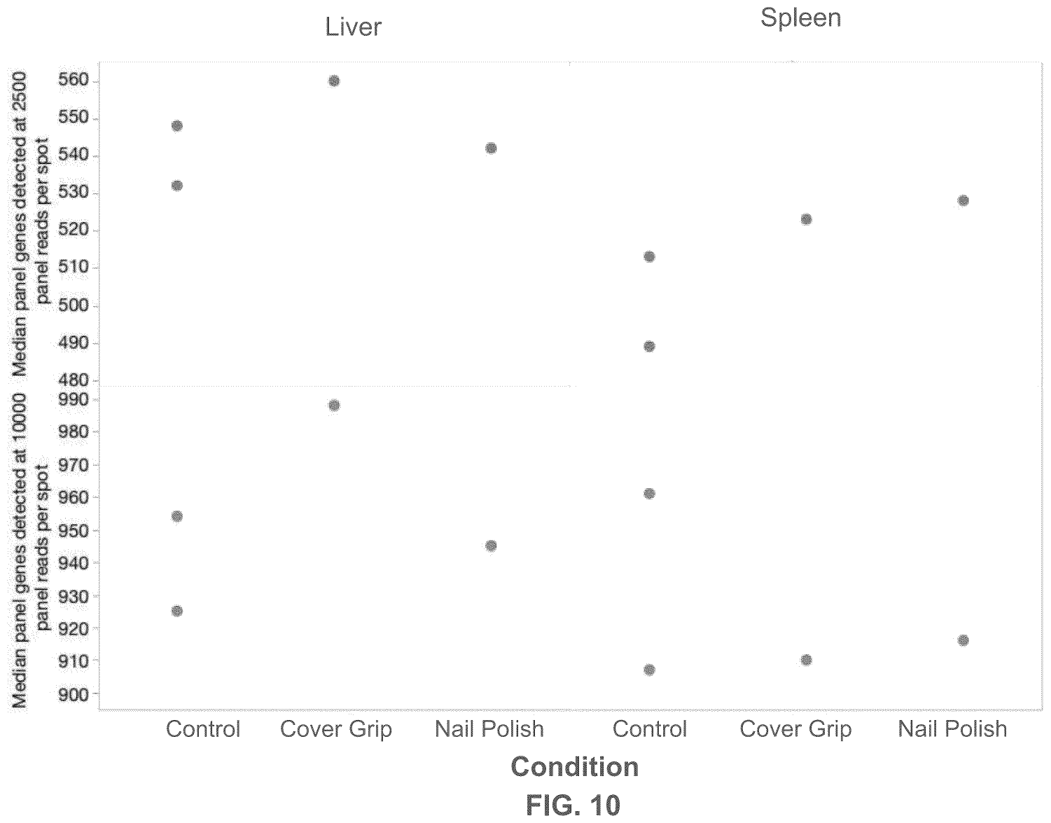

FIG. 10 shows median panel genes detected at 10,000 panel reads per spot (bottom panels) and median panel reads detected at 2,500 panel reads per spot (top panels) of liver (left panels) and spleen (right panels) tissue samples that were positioned on a substrate where an area outside of and around the tissue sample 1) had no sealant applied and was unblocked (control), 2) an area around the tissue sample was blocked with cover grip (e.g., COVERGRIP™) sealant, or 3) an area around the tissue sample was blocked with nail polish (e.g., SALLY HANSEN™). FIG. 10 shows that there was not a significant difference in gene detection from the tissue samples whether the area outside of the sample was blocked or unblocked. This result demonstrates that the sealant(s) do not have a negative effect on the workflow.

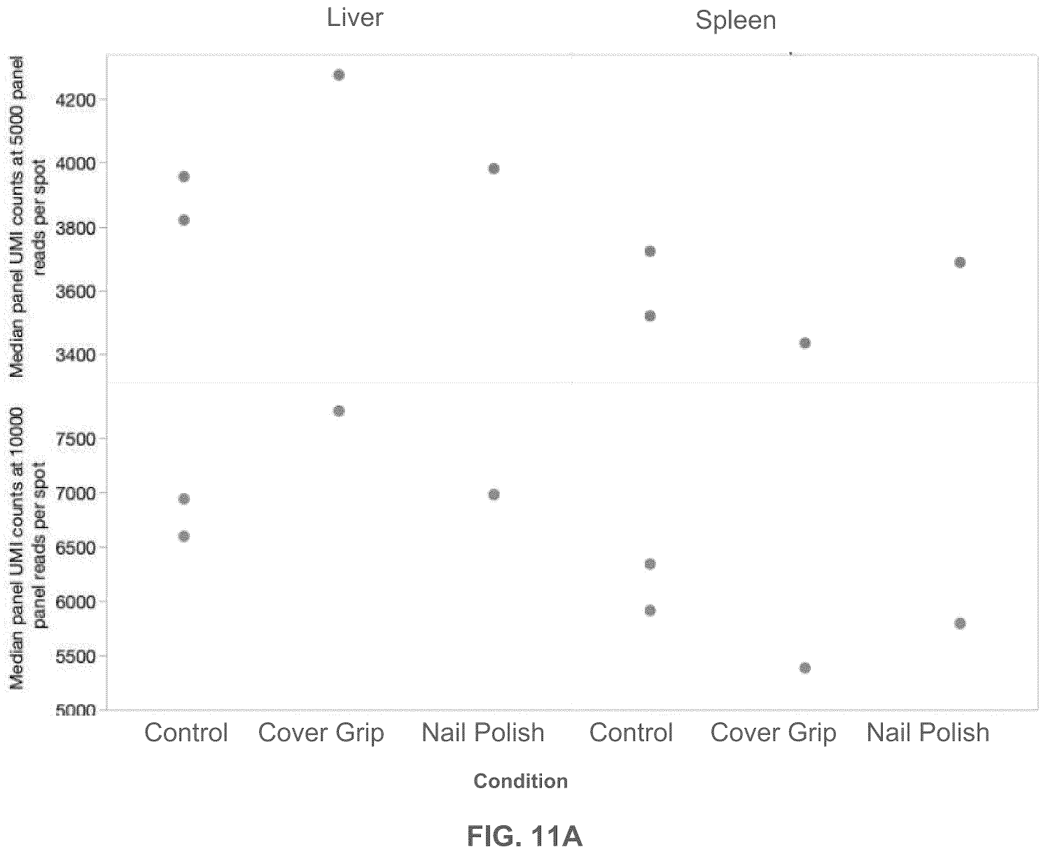

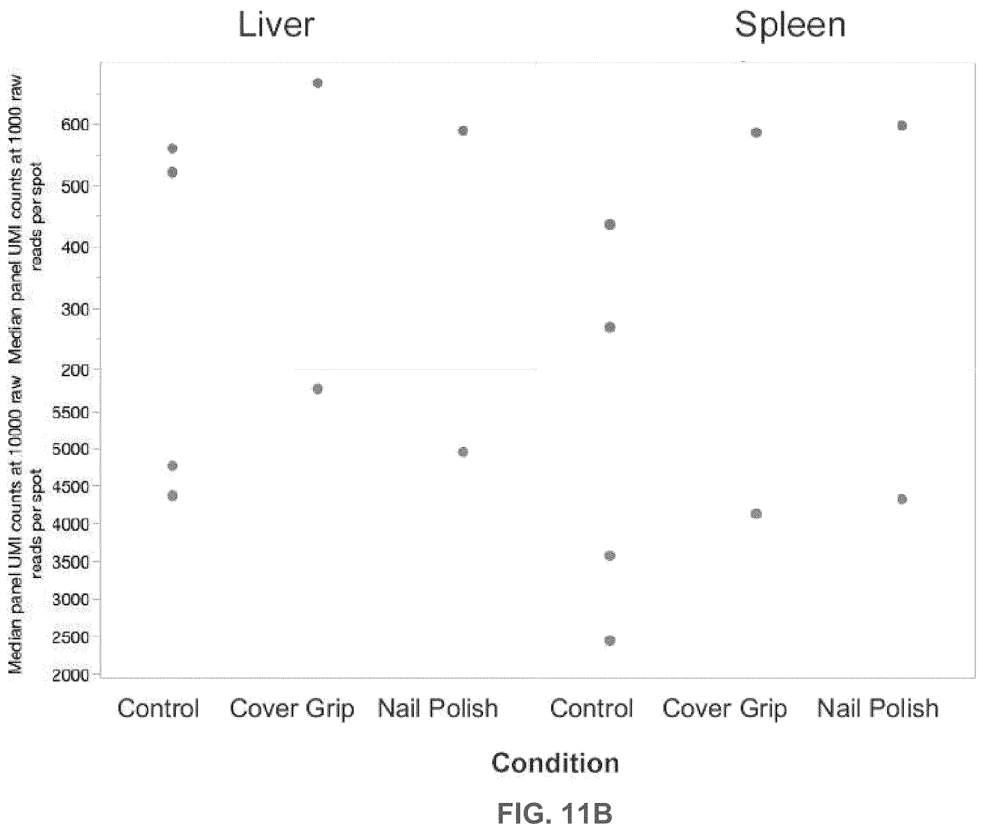

FIG. 11A shows median panel UMI counts at 10,000 panel reads per spot (bottom panels) and median panel UMI counts at 5,000 panel reads per spot for liver (left panels) and spleen (right panels) tissue samples that were positioned on a substrate where an area outside of the tissue sample (1) had no sealant applied and was unblocked (control), (2) an arca around the tissue sample was blocked with cover grip (e.g., COVERGRIP™) sealant, or (3) an area around the tissue sample was blocked with nail polish (e.g., SALLY HANSEN™). FIG. 11B shows median panel UMI counts at 10,000 raw reads per spot (bottom panels) and median panel UMI counts at 1,000 raw reads per spot for liver (left panels) and spleen (right panels) tissue samples that were positioned on a substrate where an arca outside of the tissue sample (1) had no sealant applied and was unblocked (control), (2) an area around the tissue sample was blocked with cover grip (e.g., COVERGRIP™) sealant, and or (3) an area around the tissue sample was blocked with nail polish (e.g., SALLY HANSEN™). There was no substantial difference observed in UMI counts either for panel or raw reads for the unblocked control compared to blocked tissue samples, demonstrating further that the region located under the tissue sample was unaffected by the sealant. FIGS. 11A and 11B further demonstrated that the use of sealant(s) do not have a negative effect on the tissue sample or the workflow.

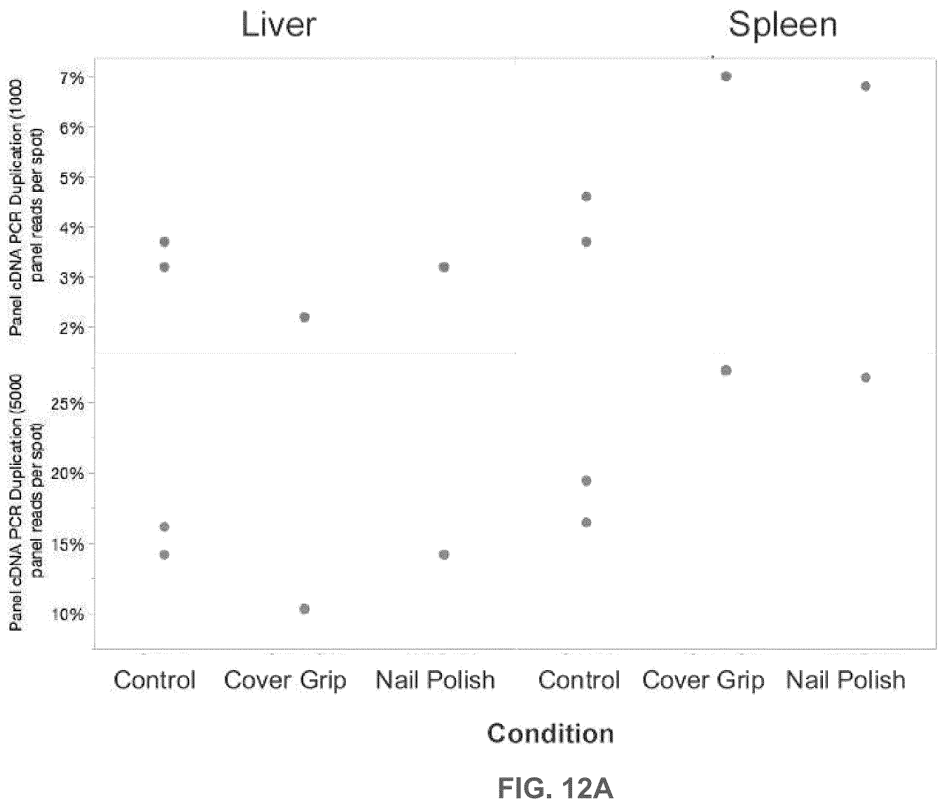

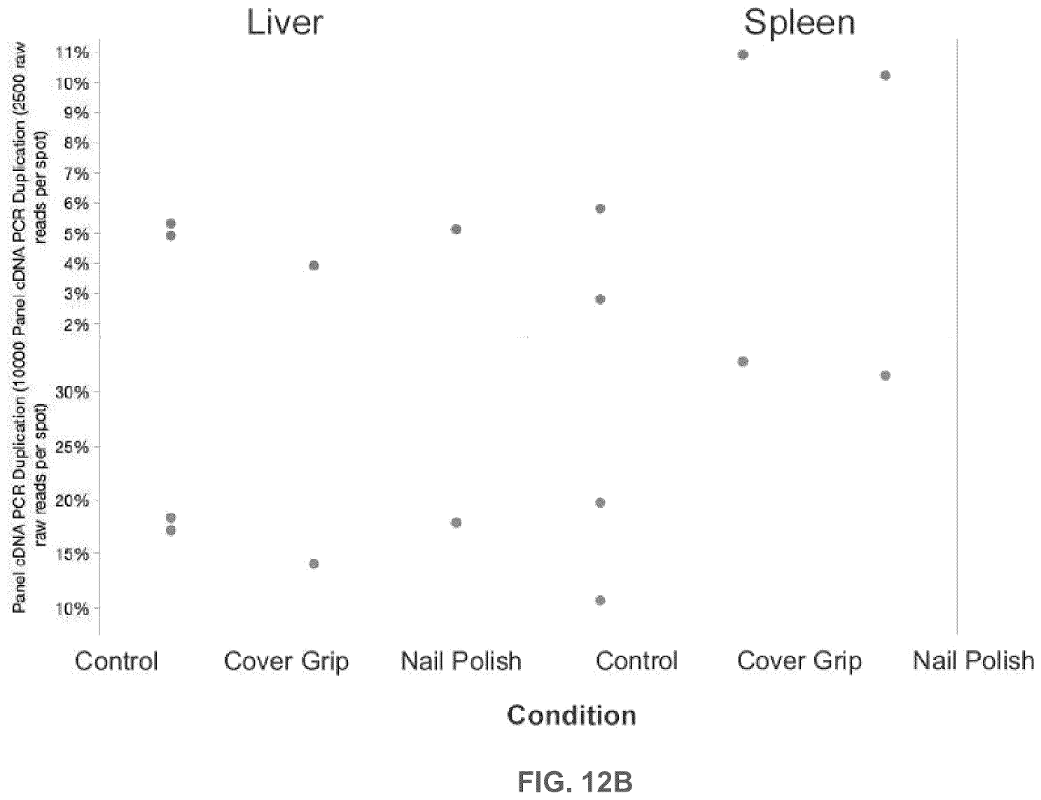

FIG. 12A shows panel cDNA PCR duplication of 5,000 panel reads per spot (bottom panels) and panel cDNA PCR duplication of 1,000 panel reads per spot (top panels) for liver (left panels) and spleen (right panels) tissue samples that were positioned on a substrate where an arca outside of the tissue sample (1) had no sealant applied and was unblocked (control), (2) an area around the tissue sample was blocked with cover grip (e.g., COVERGRIP™) sealant, or (3) an area around the tissue sample was blocked with nail polish (e.g., SALLY HANSEN™). FIG. 12B shows panel cDNA PCR duplication of 5,000 raw reads per spot (bottom panels) and panel cDNA PCR duplication of 2,500 raw reads per spot (top panels) for liver (left panels) and spleen (right panels) tissue samples that were positioned on a substrate where an arca outside of the tissue sample (1) had no sealant applied and was unblocked (control), (2) an area around the tissue sample was blocked with cover grip (e.g., COVERGRIP™) sealant, or (3) an area around the tissue sample was blocked with nail polish (e.g., SALLY HANSEN™). Comparatively, a decreased level of saturation was observed in the liver sample as compared to the spleen sample. FIGS. 12A and 12B demonstrate that despite roughly equivalent UMI counts for the controls comparative to the liver and spleen tissue samples, sequencing saturation is greater when the areas surrounding the tissue samples are blocked with a sealant.

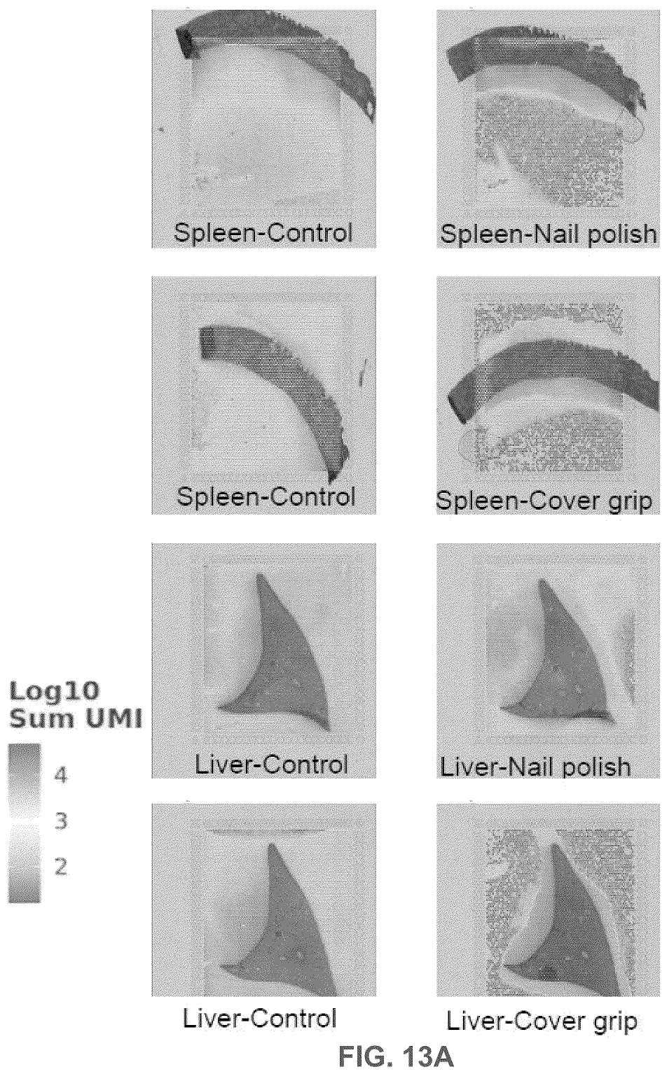

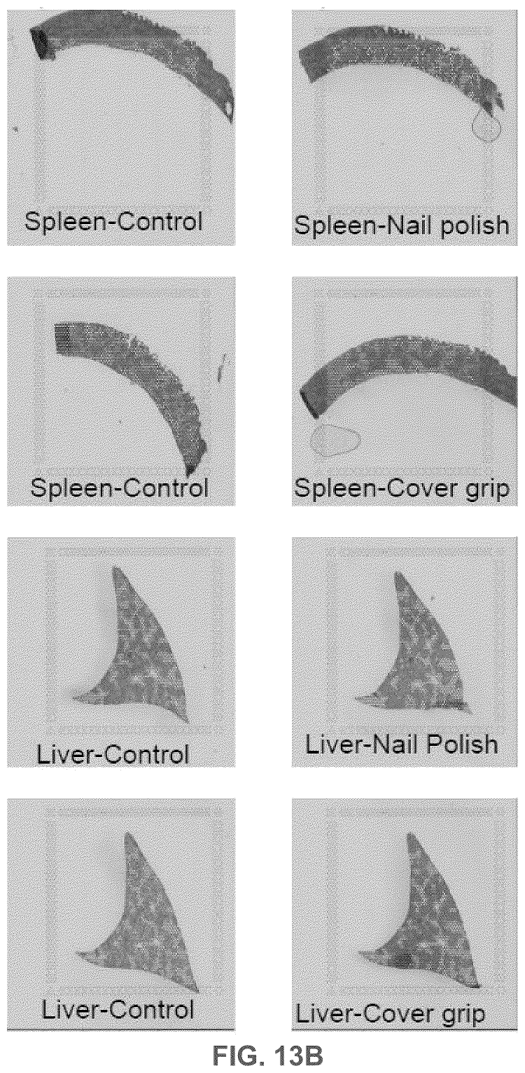

FIG. 13A shows UMI counts of spleen and liver tissue samples where an area outside of the tissue sample (1) had no sealant applied and was unblocked (control), (2) an area around the tissue sample was blocked with cover grip (e.g., COVERGRIP™) sealant, or (3) an area around the tissue sample was blocked with nail polish (e.g., SALLY HANSEN™). The top spleen control shows that one of the tissue samples moved off of the substrate during the assay (also discussed in connection with FIG. 8). The nail polish sealant of one of the ‘liver-nail polish’ tissue samples failed (also discussed in connection with FIG. 8). FIG. 13A demonstrates non-specific binding of target analytes to capture probes outside of the tissue sample in the control samples. Conversely, FIG. 13A shows decreased non-specific binding of target analytes to capture probes outside of the tissue sample in the samples where a sealant was applied around the tissue area, regardless of sealant used. As shown in FIG. 13A, red patches indicate more UMI counts and the blue patches indicates fewer UMI counts. The samples where cover grip was applied as a sealant performed slightly better than the samples where nail polish was applied, however both sealants prevented the non-specific binding of target analytes to capture probes outside of the location under the tissue sample.

FIG. 13B shows pictures of mRNA transcript clustering for spleen and liver tissue samples where, (1) no sealant was applied around the tissue sample (control), (2) the area around the tissue sample was blocked with cover grip (e.g., COVERGRIP™) sealant, or (3) the area around the tissue sample blocked with nail polish (e.g., SALLY HANSEN™). The clustering is comparable between the controls and the blocked samples, thereby supporting previous results demonstrating that the sealant(s) do not have a negative effect on workflow.

The experimental results demonstrate a sealant does not have a negative effect on the workflow for spatially capturing analytes from a biological sample. Further, that the cover grip sealant appeared to enhance assay resolution more than the nail polish sealant and the cover grip provided more consistent results. Blocking the surface of the substrate in areas outside or around the tissue sample improves sensitivity for analyte detection under the tissue sample when raw sequencing reads are observed. As such, it was determined that blocking the substrate in areas outside of the tissue sample can improve efficiency, save time, and money and can potentially impact sequencing depth for the spots under the tissue sample. The results were consistent with experimental results when a region of interest was defined and assayed after the area around that region of interest, a second region of the tissue sample and/or substrate was blocked. This assay is particularly useful for application to small samples as there is a larger area outside of the tissue sample that is uncovered by the smaller samples. Thus, the sealants described herein provide an improvement in resource conservation and a reduction and/or elimination of non-specific binding of analytes to unintended portions of the array during the assay, including regions of non-interest within a tissue sample.

The experimental results show that sealants can be used in an assay in an ad hoc manner without a negative impact on the workflow for spatially capturing analytes from a biological sample. Sealants can be applied to regions of non-interest outside of a tissue sample positioned on a substrate, and/or regions of non-interest within a tissue sample. The application of a sealant application to regions of non-interest on the substrate and/or the tissue can provide a customizable advantage without increasing a burden to the workflow.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-20. (canceled)

21. A composition comprising:
a substrate comprising a plurality of capture probes affixed to a surface of the substrate;
a tissue section in contact with the plurality of capture probes; and
a liquid sealant applied to an area on: (i) the surface outside or surrounding the tissue section and/or (ii) the tissue section or a portion thereof.

22. The composition of claim 21, wherein the liquid sealant prevents permeabilization of the tissue section within the sealed area or release of an analyte from the tissue section.

23. The composition of claim 21, wherein the liquid sealant prevents interaction of one or more capture probes of the plurality of capture probes with the tissue section or an analyte thereof.

24. The composition of claim 21, wherein the liquid sealant is applied to the substrate surface outside or surrounding the tissue section, thereby forming a hydrophobic seal around the tissue section.

25. The composition of claim 21, wherein the liquid sealant comprises a hydrophobic sealant, an organic adhesive, one or more polymer resins, nitrocellulose, ethyl acetate, butyl acetate, and/or a coverslip sealant.

26. The composition of claim 21, wherein the liquid sealant is a coverslip sealant, a liquid coverslip, nail polish, liquid from a hydrophobic pen, a mounting media, a gel, an adhesive, a bilayer, or a combination thereof.

27. The composition of claim 21, further comprising a permeabilization agent comprising an organic solvent, a detergent, an enzyme, or a combination thereof.

28. The composition of claim 27, wherein the permeabilization agent comprises an endopeptidase, a protease sodium dodecyl sulfate (SDS), polyethylene glycol tert-octylphenyl ether, polysorbate 80, and polysorbate 20, N-lauroylsarcosine sodium salt solution, saponin, a nonionic surfactant, or polyoxyethylene sorbitol.

29. The composition of claim 28, wherein the endopeptidase is pepsin or proteinase K.

30. The composition of claim 21, further comprising a removing agent that removes the liquid sealant from the sealed area.

31. The composition of claim 30, wherein the removing agent comprises a solvent, an acid, a base, a buffer, or a combination thereof.

32. The composition of claim 21, wherein each of the plurality of capture probes identifies a location on the surface of the substrate.

33. The composition of claim 21, wherein each of the plurality of capture probes comprises a spatial barcode and a capture domain that captures an analyte or a proxy thereof from the tissue section.

34. The composition of claim 33, wherein the capture domain comprises a poly(T) sequence

35. The composition of claim 33, wherein the capture domain captures an RNA molecule from the tissue section.

36. The composition of claim 33, wherein the capture domain captures a cDNA molecule generated from an RNA molecule from the tissue section.

37. The composition of claim 21, wherein each of the plurality of capture probes further comprises a cleavage domain, a functional sequence, a unique molecular identifier (UMI), or a combination thereof.

38. The composition of claim 21, wherein the tissue section is a fresh tissue section, a frozen tissue section, a formalin fixed paraffin embedded tissue section, or a fixed tissue section.

39. The composition of claim 21, wherein the tissue section is previously stained using immunofluorescence, immunohistochemistry, hematoxylin, or eosin.

40. The composition of claim 21, wherein the substrate is a glass slide.
